(19) United States
(12) Patent Application Publication
(10) Pub. No.: US 2024/0252349 A1
(43) Pub. Date: Aug. 1, 2024

(54) COLD SENSATION PRESENTATION DEVICE AND PROGRAM

(71) Applicant: University of Tsukuba, Tsukuba-shi, Ibaraki (JP)

(72) Inventors: Yoshihiro Kuroda, Tsukuba-shi (JP); Jiayi Xu, Tsukuba-shi (JP)

(21) Appl. No.: 18/564,538

(22) PCT Filed: May 30, 2022
(86) PCT No.: PCT/JP2022/021922

§ 371 Date: Nov. 27, 2023

(30) Foreign Application Priority Data
May 31, 2021 (JP) 2021-091472

(51) Int. Cl. A61F 7/00

(52) U.S. Cl. CPC A61F 7/00; A61F 2007/0052; A61F 2007/006; A61F 2007/0063; A61F 2007/0093

(57) ABSTRACT

A cold sensation presentation device includes: a cold stimulus section that contactlessly gives a cold stimulus to a skin; a warm stimulus section that contactlessly gives a warm stimulus to a skin; and a control unit that presents a cold sensation by causing a temporal change in the intensity of the cold stimulus in a cold stimulus state where the intensity of the warm stimulus is relatively small and a temporal change in the intensity of the warm stimulus in a warm stimulus state where the intensity of the warm stimulus is relatively large to be different from each other, and repeating the cold stimulus state and the warm stimulus state in a state of keeping the intensity of the cold stimulus constant.

### TECHNICAL FIELD

The present invention relates to a cold sensation presentation device and a program.

### BACKGROUND ART

A device that gives a thermal stimulus to the skin of a subject has been proposed (e.g., Patent Document 1).

### CITATION LIST

### Patent Document

Patent Document 1: JP 2020-22617 A

### SUMMARY OF INVENTION

### Technical Problem

It is preferable that the device that gives such a thermal stimulus can continuously give the subject a natural sense of being in a real environment while artificially reproducing the real environment.

However, according to the technique as described above, since a thermal stimulus is given by being brought into contact with the skin of the subject, a natural sense cannot be given in some cases.

The present invention has been made in view of the above points, and provides a cold sensation presentation device and a program that can give a persistent cold sensation close to a real environment.

### Solution to Problem

One aspect of the present invention is directed to a cold sensation presentation device including: a cold stimulus section that contactlessly gives a cold stimulus to a skin; a warm stimulus section that contactlessly gives a warm stimulus to a skin; and a control unit that presents a cold sensation by causing a temporal change in the intensity of the cold stimulus in a cold stimulus state where the intensity of the warm stimulus is relatively small and a temporal change in the intensity of the warm stimulus in a warm stimulus state where the intensity of the warm stimulus is relatively large to be different from each other, and repeating the cold stimulus state and the warm stimulus state in a state of keeping the intensity of the cold stimulus constant.

One aspect of the present invention is directed to the cold sensation presentation device in which the control unit keeps the intensity of the cold stimulus constant by keeping the temperature and the flow rate of cold air constant.

One aspect of the present invention is directed to the cold sensation presentation device in which the control unit temporally changes the intensity of the warm stimulus from a relatively small state to a relatively large state in the warm stimulus state.

One aspect of the present invention is directed to the cold sensation presentation device in which the control unit ends cold sensation presentation by gradually decreasing the intensity of the cold stimulus and the intensity of the warm stimulus or gradually decreasing presentation time of the cold stimulus and presentation time of the warm stimulus.

One aspect of the present invention is directed to the cold sensation presentation device in which among conduction, convection, and radiation, which are types of heat transfer methods, a type with which the cold stimulus section gives the cold stimulus and a type with which the warm stimulus section gives the warm stimulus are different from each other.

One aspect of the present invention is directed to a program for causing a computer that controls a cold sensation presentation device including a cold stimulus section that contactlessly gives a cold stimulus to a skin and a warm stimulus section that contactlessly gives a warm stimulus to a skin to execute causing a temporal change in the intensity of the cold stimulus in a cold stimulus state where the intensity of the warm stimulus is relatively small and the temporal change in the intensity of the warm stimulus in a warm stimulus state where the intensity of the warm stimulus is relatively large to be different from each other, and repeating the cold stimulus state and the warm stimulus state in a state of keeping the intensity of the cold stimulus constant.

### Advantageous Effects of Invention

According to the present invention, it is possible to give a persistent cold sensation close to a real environment.

### BRIEF DESCRIPTION OF DRAWINGS

FIG. 1 is a view illustrating an example of a configuration of a cold sensation presentation device of the present embodiment.

FIG. 2 is a view illustrating an example of a configuration of a cold air generation unit of the present embodiment.

FIG. 3 is a view illustrating an example of a configuration of a presentation unit of the present embodiment.

FIG. 4 is a view illustrating an example of a configuration of a light source of the present embodiment.

FIG. 5 is a graph showing an example of human perception of temperature changes.

FIG. 6 is a view illustrating an example of a method of giving a warm or cold stimulus by the cold sensation presentation device of the present embodiment.

FIG. 7 is a flowchart showing an example of operation of a control unit of the present embodiment.

FIG. 8 is a diagram showing an example of control by the control unit of the present embodiment.

FIG. 9 is a graph showing an example of temporal change in temperature of skin when temporal changes in the intensity of a cold stimulus and the intensity of a warm stimulus are given by the control unit of the present embodiment.

FIG. 10 is a diagram showing an example of an experimental result by the cold sensation presentation device of the present embodiment.

FIG. 11 is a diagram showing an example of an experimental result in a case of temporally changing a cold air jet.

FIG. 12 is a diagram showing a variation of control by the control unit of the present embodiment.

### DESCRIPTION OF EMBODIMENTS

### Background Art

Warm or cold sensation, which is a type of cutaneous sensation, plays a role of recognizing a material and a state of an object a person touches, and also plays a role of recognizing cold and warm of an environment in which the person is. Therefore, there is a possibility that a warm or cold sensation display artificially reproduces and expands a real object or an environment. Researches of transmitting thermal characteristics of an object by a contact type display using a Peltier element have been actively conducted, but if contactless warm or cold sensation control is achieved, it becomes possible to provide a sense of going to an environment such as the south pole or a tropical rainforest. Furthermore, if persistent warm or cold sensation can be provided, the sense of being in the environment becomes possible, and it is expected to lead to achievement of new sense and affect control.

In order to reproduce warm or cold sensation in a state of being immersed in the environment, not instantaneous (short time) but persistent warm or cold sensation provision is required. Many related arts are limited to instantaneous warm or cold sensation provision.

For example, instantaneous contact warm or cold sensation provision using cold water or warm water and instantaneous contactless warm or cold sensation provision using a light emitting diode (LED) are performed. An instantaneous contactless cold sensation control technique using an ultra-low temperature cold air jet has been achieved.

Hereinafter, embodiments of the present invention will be described with reference to the drawings.

### Embodiments

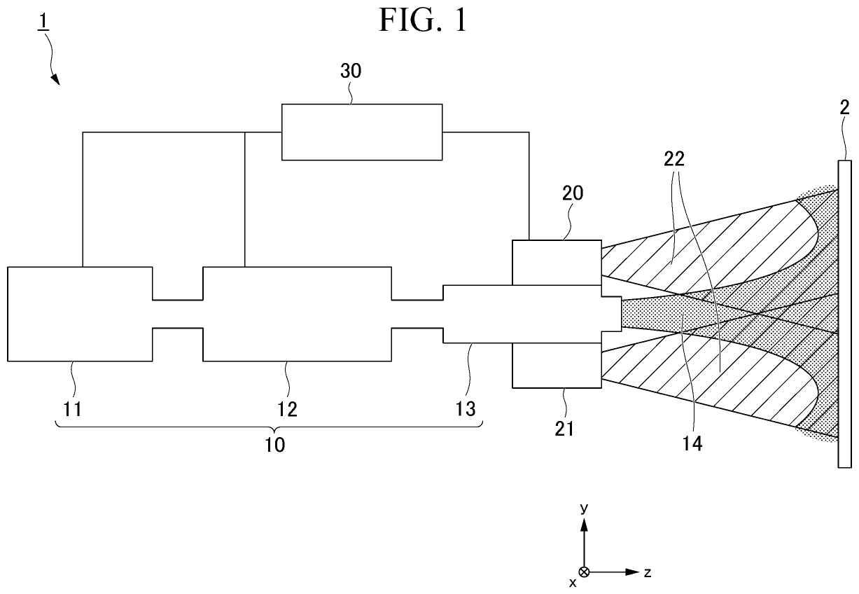

FIG. 1 is a view illustrating an example of a configuration of a cold sensation presentation device 1 of the present embodiment. In the following, where necessary, description will be given using a three-dimensional orthogonal coordinate system of the x axis, the y axis, and the z axis.

The cold sensation presentation device 1 includes a cold stimulus section 10, a warm stimulus section 20, and a control unit 30.

The control unit 30 includes, for example, a computer that operates with a predetermined program, and controls each section of the cold sensation presentation device 1.

The cold stimulus section 10 includes a cold air generation unit 11, a flow rate control unit 12, and a presentation unit 13.

The cold air generation unit 11 generates cold air with a predetermined flow rate. The flow rate control unit 12 controls a flow rate of cold air generated by the cold air generation unit 11.

The flow rate control unit 12 controls a flow rate of cold air generated by the cold air generation unit 11. As an example, the flow rate control unit 12 includes a solenoid valve. The flow rate control unit 12 controls the flow rate of the cold air 14 by changing an opening degree of the solenoid valve to an opening degree corresponding to a pulse width modulation (PWM) current applied from the control unit.

The presentation unit 13 discharges, in the z axis direction, the cold air 14 whose flow rate has been controlled by flow rate control unit 12.

The warm stimulus section 20 includes a light source 21. The light source 21 emits light 22 having the intensity corresponding to the control of the control unit 30. The warm stimulus section 20 may include a plurality of the light sources 21.

Hereinafter, the configuration of each unit will be described more specifically.

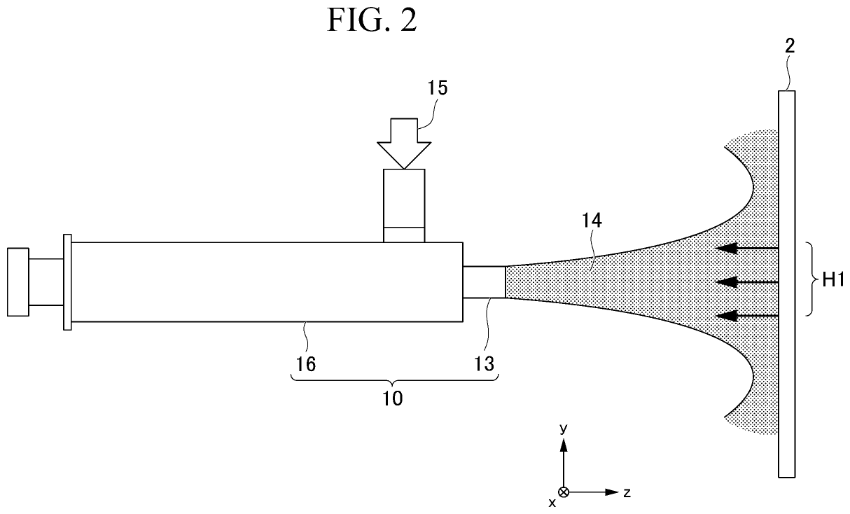

FIG. 2 is a view illustrating an example of a configuration of the cold air generation unit 11 of the present embodiment.

As an example, the cold air generation unit 11 includes a vortex tube 16. The vortex tube 16 separates compressed air 15 supplied from an air compressor (not illustrated) into warm air and cold air by making the compressed air 15 a vortex flow. Of the warm air and the cold air separated in the vortex tube 16, the cold air generation unit 11 guides the cold air to the presentation unit 13 via the flow rate control unit 12 (not illustrated in FIG. 2).

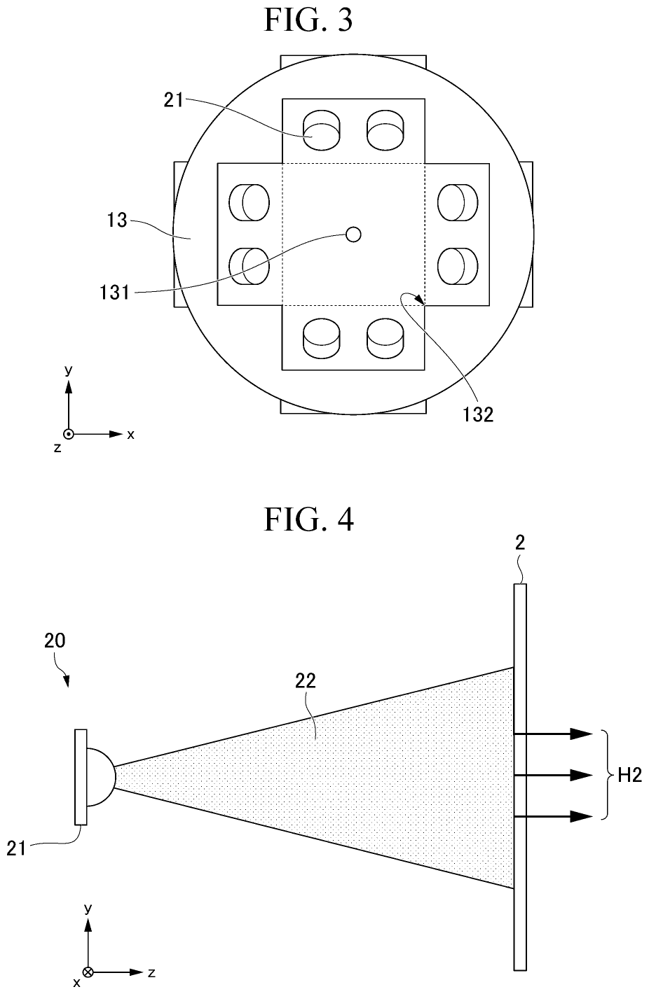

FIG. 3 is a view illustrating an example of the configuration of the presentation unit 13 of the present embodiment.

The presentation unit 13 includes a cold air discharge port 131 at a substantially center position on the xy plane. The cold air discharge port 131 discharges the cold air 14 in the z axis direction. For example, the cold air 14 is discharged toward skin 2 to take heat H1 from the skin 2.

That is, the cold stimulus section 10 contactlessly gives a cold stimulus to the skin 2.

The presentation unit 13 may include a wall portion 132 having a height in the z axis direction around the cold air discharge port 131. This wall portion 132 deters the cold air 14 discharged from the cold air discharge port 131 from diffusing in the xy plane direction.

FIG. 4 is a view illustrating an example of the configuration of the light source 21 of the present embodiment.

The light source 21 is, for example, a light emitting diode (LED), and emits the light 22 in the same direction (i.e., in the z axis direction) as the discharge direction (z axis direction) of the cold air 14. The light 22 is emitted toward the skin 2, for example, and gives heat H2 to the skin 2.

That is, the warm stimulus section 20 contactlessly gives a warm stimulus to the skin 2.

As illustrated in FIG. 3, the plurality of light sources 21 may be disposed around the cold air discharge port 131 of the presentation unit 13.

In the cold sensation presentation device 1, the type of the heat transfer method with which the cold stimulus section 10 gives the cold stimulus and the type of the heat transfer method with which the warm stimulus section 20 gives the warm stimulus are different from each other.

Generally, the heat transfer method is classified into three types of conduction, convection, and radiation. As described above, the cold stimulus section 10 gives a cold stimulus by discharge (i.e., conduction or convection) of the cold air 14. The warm stimulus section 20 gives a warm stimulus by irradiation with the light 22 (i.e., radiation).

According to the cold sensation presentation device 1 thus configured, the cold stimulus given by the cold stimulus section 10 and the warm stimulus given by the warm stimulus section 20 do not interfere with each other in a space, and the cold sensation presentation device 1 can give stimuli independently of each other. Therefore, according to the cold sensation presentation device 1 thus configured, it is possible to more easily control the intensity of the cold stimulus and the intensity of the warm stimulus, ignoring a mutual influence of the cold stimulus and the warm stimulus given to the temperature of the skin 2.

### Time Division Provision of Contactless Warm or Cold Stimulus

Time division provision of the contactless warm or cold stimulus will be described. Note that in the description of the present embodiment, “provision” and “presentation” are synonymous.

For temperature sense of the skin 2, there are two types of reactions, static reaction and dynamic reaction. The static reaction is to react to an actual temperature of the skin surface. If the temperature of the skin surface can be greatly changed, a temperature sense can be generated by the static reaction. However, in a case of providing a temperature sense by the static reaction, it takes a long time for the sense to occur, which is not desirable.

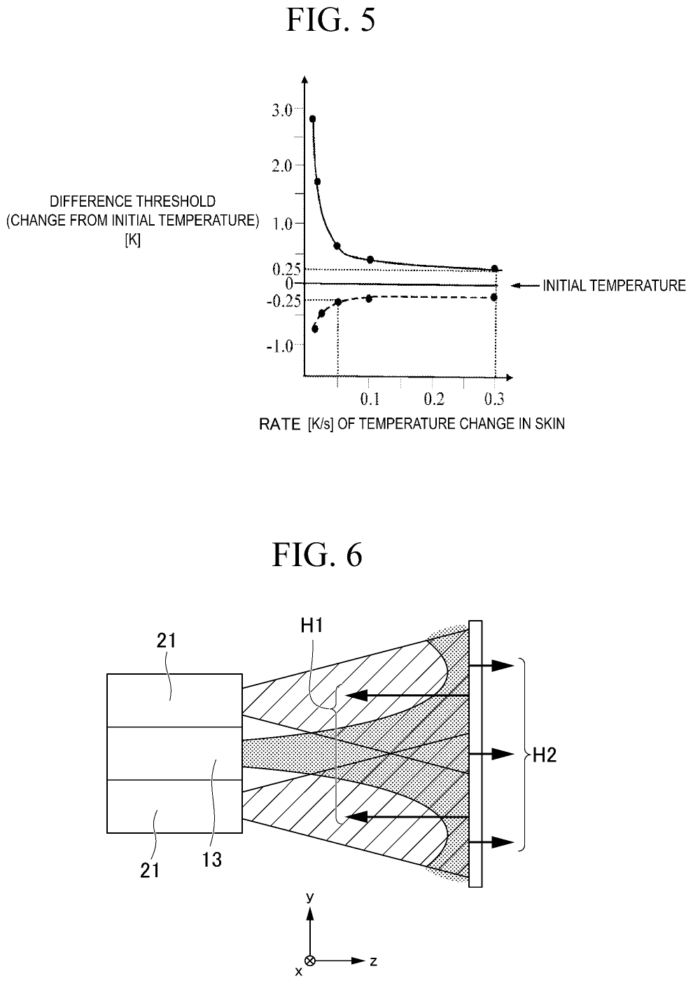

On the other hand, the dynamic reaction is to react to the rate of a temperature change. An example of the dynamic reaction of the skin 2 is shown in FIG. 5.

FIG. 5 is a diagram showing an example of the human perception of temperature change of the skin 2. As shown in FIG. 5, when the temperature changes rapidly, the skin 2 can perceive a small temperature change. When the temperature changes slowly, the skin 2 does not have a temperature sense until a large temperature change occurs. The higher the rate of temperature change is, the shorter the time in which temperature sense occurs, and the skin 2 perceives a strong stimulus.

FIG. 6 is a view illustrating an example of a method of giving a warm or cold stimulus by the cold sensation presentation device 1 of the present embodiment.

In the present embodiment, a cold stimulus stronger than a perception threshold is provided to the skin 2, and a warm stimulus weaker than the perception threshold is provided to the skin 2. Repeating two steps of giving the strong cold stimulus and returning the skin temperature to the original temperature by the weak warm stimulus achieves time division provision of the warm or cold stimulus. When the temperature of the skin is changed within a range of 0.25 [C], a warm stimulus with the rate of temperature change of less than 0.3 [° C./s] is not preceived, and a cold stimulus with a rate of temperature change of equal to or greater than 0.05 [° C./s] is precerived.

The cold sensation presentation device 1 of the present embodiment sustains a cold sensation in the skin 2 by using asymmetry between the rate of temperature change at which the skin 2 perceives a warm stimulus and the rate of temperature change at which the skin 2 perceives a cold stimulus.

### Operation of Control Unit

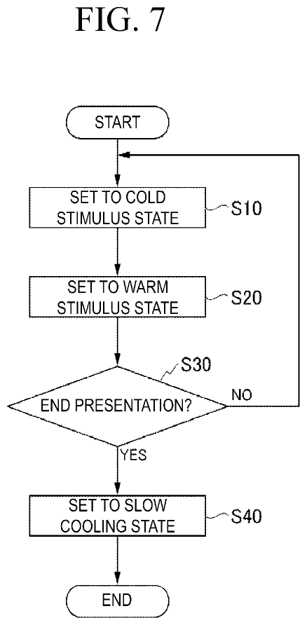

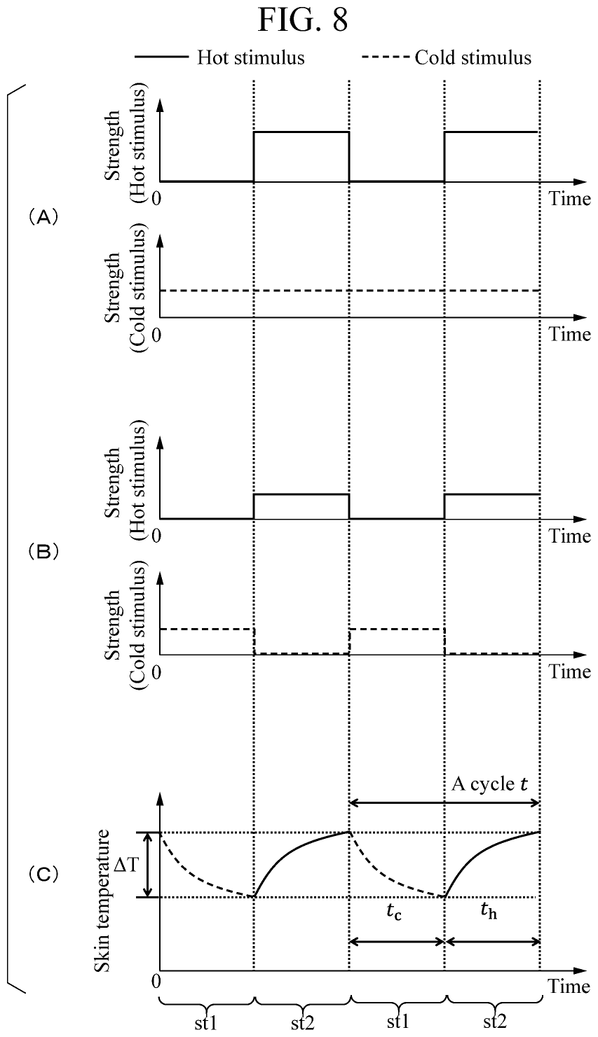

FIG. 7 is a flowchart showing an example of the operation of the control unit 30 of the present embodiment. As described above, the control unit 30 sustains the cold sensation in the skin 2 by alternately repeating a cold stimulus state (e.g., a cold stimulus state st1 shown in FIG. 8) and a warm stimulus state (e.g., a warm stimulus state st2 shown in FIG. 8).

Depending on individual differences in temperature of the skin 2 of the subject in a situation where no thermal stimulus is given, individual differences may occur in how the cold sensation is perceived on the skin 2. Therefore, it is preferable to perform calibration for the cold stimulus and the warm stimulus for each subject to obtain the temperature serving as the reference of control. In this case, it is preferable that a storage device (not illustrated) store a calibration result (e.g., reference temperature for each individual), and the control unit 30 calculate the intensity of the cold stimulus and the intensity of the warm stimulus based on this reference temperature.

(Step S10) The control unit 30 sets the cold stimulus state st1. In the cold stimulus state st1, the control unit 30 causes the presentation unit 13 to discharge the cold air 14 having a predetermined temperature and a predetermined flow rate. In the cold stimulus state st1, the control unit 30 may cause the light 22 to be emitted from the light source 21 while causing the cold air 14 to be discharged.

(Step S20) The control unit 30 sets the warm stimulus state st2. In the warm stimulus state st2, the control unit 30 causes the light 22 of a predetermined light amount to be emitted from the light source 21. In the warm stimulus state st2, the control unit 30 may cause the cold air 14 to be discharged while causing the light 22 to be emitted from the light source 21.

FIG. 8 is a diagram showing an example of control by the control unit 30 of the present embodiment.

FIG. 8[A] individually shows a presentation pattern of the cold stimulus by the cold stimulus section 10 and a presentation pattern of the warm stimulus by the warm stimulus section 20.

FIG. 8[B] shows a result of combining the presentation pattern of the cold stimulus by the cold stimulus section 10 and the presentation pattern of the warm stimulus by the warm stimulus section 20.

In this example, the control unit 30 keeps a discharge temperature and a discharge amount of the cold air 14 by the cold stimulus section 10 constant in the cold stimulus state st1 and the warm stimulus state st2. The control unit 30 decreases the emission amount of the light 22 by the warm stimulus section 20 (weakens the light 22) in the cold stimulus state st1, and increases the emission amount of the light 22 by the warm stimulus section 20 (strengthens the light 22) in the warm stimulus state st2.

In the cold stimulus state st1, since the warm stimulus is not presented and the cold stimulus is presented, the result of combining the warm stimulus and the cold stimulus is the intensity of only the cold stimulus.

In the warm stimulus state st2, since the warm stimulus and the cold stimulus are simultaneously presented, in the result of combining the warm stimulus and the cold stimulus, a temperature decrease due to the cold stimulus is canceled and a small temperature increase remains.

As a result of presentation of the cold stimulus and the warm stimulus shown in FIG. 8[A] and FIG. 8[B], the temperature of the skin changes as shown in FIG. 8[C].

Here, the cold stimulus state st1 is a state where the intensity of the warm stimulus is relatively small. The warm stimulus state st2 is a state where the intensity of the warm stimulus is relatively large. In other words, the intensity of the warm stimulus in the cold stimulus state st1 is smaller than the intensity of the warm stimulus in the warm stimulus state st2.

The intensity of the warm stimulus being relatively small includes stopping presentation of the warm stimulus (i.e., making the intensity of the warm stimulus zero) as shown in FIG. 8[A].

The intensity of the warm stimulus being relatively high includes presentation of the warm stimulus while continuously presenting the cold stimulus (i.e., continuously discharging the cold air 14) as shown in FIG. 8[A].

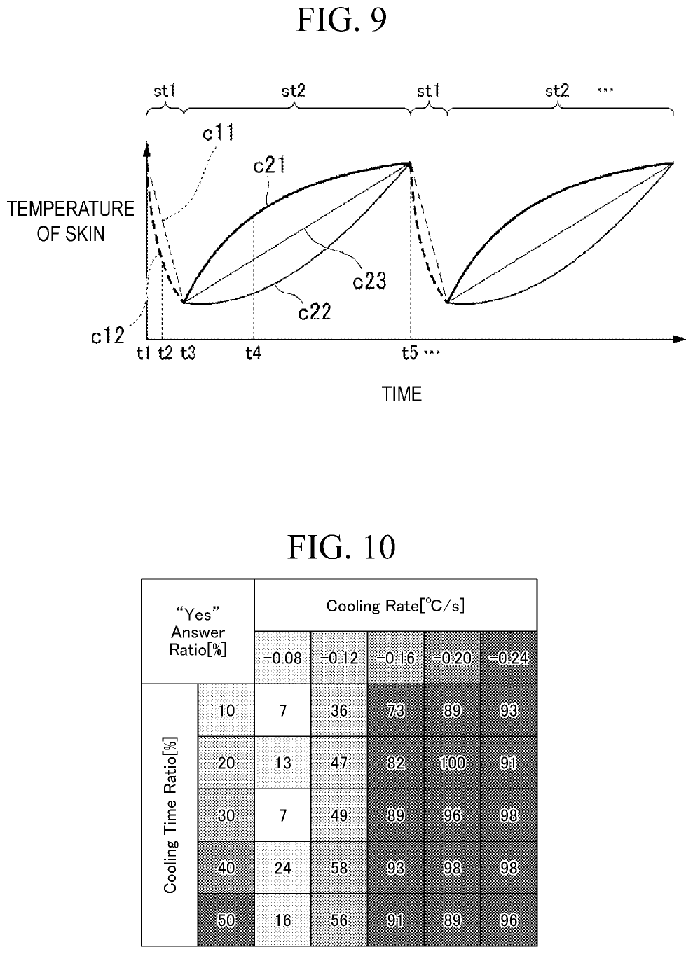

The temporal change of the temperature of the skin shown in FIG. 8[C] will be described in more detail with reference to FIG. 9.

FIG. 9 is a diagram showing an example of temporal change in temperature of skin when temporal changes in the intensity of a cold stimulus and the intensity of a warm stimulus are given by the control unit 30 of the present embodiment.

The control unit 30 presents a cold sensation by causing a temporal change in the intensity of the cold stimulus in the cold stimulus state st1 and a temporal change in the intensity of the warm stimulus in the warm stimulus state st2 to be different from each other and repeating the cold stimulus state st1 and the warm stimulus state st2.

As described above, in the cold stimulus state st1, the control unit 30 may cause the light 22 to be emitted from the light source 21 while causing the cold air 14 to be discharged. In the warm stimulus state st2, the control unit 30 may cause the cold air 14 to be discharged while causing the light 22 to be emitted from the light source 21.

In FIG. 9, a period from time t1 to time t3 is the cold stimulus state st1, and a period from time t3 to time t5 is the warm stimulus state st2. After time t5, the cold stimulus state st1 and the warm stimulus state st2 are alternately repeated.

FIG. 9 shows a graph of temporal changes in the temperature of the skin in the cold stimulus state st1 and the warm stimulus state st2. As an example, when the intensity of the cold stimulus is temporally changed from a small state to a large state so that the rate of temperature change in the skin becomes constant, it is represented linearly as a curve c11.

Here, in the cold stimulus state st1, the control unit 30 may temporally change the intensity of the cold stimulus from a relatively small state to a relatively large state.

As an example, the control unit 30 makes the intensity of the cold stimulus from time t2 to time t3 larger than the intensity of the cold stimulus from time t1 to time t2. In this case, the temperature of the skin temporally changes as indicated by the curve c11. According to the cold sensation presentation device 1 thus configured, a weaker cold stimulus is given to the skin 2 immediately after the cold stimulus starts to be provided to the skin 2 (e.g., between time t1 and time t2) as compared with the subsequent period (e.g., between time t2 and time t3). As a result, the cold sensation presentation device 1 can cause a constant cold sensation (i.e., a stable cold sensation) to be felt by the skin 2, for example, as compared with the case where the temperature of the skin is not temporally changed as shown by a curve c12.

Note that the control unit 30 needs not temporally change the intensity of the cold stimulus in the cold stimulus state st1.

The control unit 30 may temporally change the intensity of the warm stimulus from a relatively small state to a relatively large state in the warm stimulus state st2.

As an example, the control unit 30 makes the intensity of the warm stimulus from time t3 to time t4 smaller than the intensity of the warm stimulus from time t4 to time t5. In this case, the temperature of the skin temporally changes as indicated by a curve c23 or a curve c22. According to the cold sensation presentation device 1 thus configured, a weaker warm stimulus is given to the skin 2 immediately after the warm stimulus starts to be provided to the skin 2 (e.g., between time t3 to time t4) as compared with the subsequent period (e.g., between time t4 to time t5). As a result, the cold sensation presentation device 1 can cause the skin 2 to be less likely to feel a warm stimulus, and can cause the cold sensation to be more likely to be perceived to continue.

Note that the control unit 30 needs not temporally change the intensity of the warm stimulus in the warm stimulus state st2.

As an example, the control unit 30 does not temporally change the intensity of the warm stimulus from time t3 to time t5. In this case, the temperature of the skin temporally changes as indicated by a curve c21. According to the cold sensation presentation device 1 thus configured, it is possible to further simplify the control of the intensity of the warm stimulus.

According to the cold sensation presentation device 1 thus configured, the output performance limit of the warm stimulus section 20 may be low as compared with a case where the intensity of the warm stimulus is temporally changed, and the device can be simplified.

The intensity of the cold stimulus may be changed by changing the temperature of the fluid (cold air 14), the flow rate of the fluid, the presentation time, the energy density per unit time, and the like.

The intensity of the warm stimulus may be changed by changing the current value supplied to the light source 21, the number of light sources 21, the presentation time, the energy density per unit time, and the like.

Returning to FIG. 7, the description of the operation of the control unit 30 will be continued.

(Step S30) The control unit 30 determines whether or not to end presentation of the stimulus. If the control unit 30 determines to continue presentation of the stimulus (step S30; NO), the process returns to step S10 to repeat the cold stimulus state st1 and the warm stimulus state st2. If the control unit 30 determines to end presentation of the stimulus (step S30; YES), the process proceeds to step S40.

(Step S40) The control unit 30 sets a slow cooling state st3, and then ends the process. Here, the slow cooling state st3 is a state where the intensity of the cold stimulus and the intensity of the warm stimulus are gradually decreased to end the cold sensation presentation. That is, the control unit 30 gradually decreases the intensity of the cold stimulus and the intensity of the warm stimulus, and ends the cold sensation presentation. According to the cold sensation presentation device 1 thus configured, it is possible to cause the cold stimulus to be perceived to have disappeared more naturally in the skin 2 as compared with a case where the cold sensation presentation is ended without gradually decreasing the intensity of the cold stimulus and the intensity of the warm stimulus.

Note that in the slow cooling state st3, the control unit 30 may gradually shorten provision times tc and th (i.e., gradually decrease a temperature change ΔT) with the intensity of the cold stimulus and the intensity of the warm stimulus constant.

### Summary of Embodiments

As described above, in the cold sensation presentation device 1 of the present embodiment, both the cold stimulus section 10 and the warm stimulus section 20 contactlessly give a thermal stimulus to the skin. Therefore, according to the cold sensation presentation device 1 of the present embodiment, it is possible to hygienically present a thermal stimulus in an environment closer to nature as compared with a case of giving a thermal stimulus in contact with the skin. According to the cold sensation presentation device 1 of the present embodiment, since it is not affected by the contact of the skin, it is easy to evaluate the characteristics of a human with respect to stimuli, and it is possible to easily examine the mechanism by which sense occurs.

In the cold sensation presentation device 1 of the present embodiment, the control unit 30 repeats the cold stimulus state st1 and the warm stimulus state st2, whereby the cold sensation is presented with the spatial mean temperature fluctuating in a small range.

Here, when a cold stimulus is continuously given to the skin as in related arts, there have been cases of such problems that the sense of the skin becomes dull with the lapse of time or that the skin temperature excessively drops.

According to the cold sensation presentation device 1 of the present embodiment, the sense of the skin is less likely to become dull and excessive drop of the skin temperature can be reduced, and the cold sensation can be continuously given as compared with the case of continuously giving the cold stimulus.

Note that in the above embodiment, in the above embodiment, the description has been given on an assumption that the cold stimulus section 10 discharges the cold air 14, but the present disclosure is not limited to this. The cold stimulus section 10 may give a cold stimulus by, for example, transpiration action of atomized water (e.g., mist), alcohol, or the like.

The description has been given on an assumption that the warm stimulus section 20 includes the light source 21 including the LED of the visible light band, but the present invention is not limited to this. The warm stimulus section 20 only needs to contactlessly give a warm stimulus to the skin 2, and may also give a warm stimulus by discharge of warm air or irradiation with infrared rays, for example.

As described above, the cold sensation presentation device 1 of the present embodiment controls a temporal change rate in the target temperature on the skin of the subject by giving a warm stimulus and a cold stimulus to the subject. At this time, the cold sensation presentation device 1 presents a cold sensation by causing a temporal change in the intensity of the cold stimulus and a temporal change in the intensity of the warm stimulus to be different from each other and repeating the cold stimulus state and the warm stimulus state in a state of keeping the intensity of the cold stimulus constant.

For example, the cold sensation presentation device 1 keeps the intensity of the cold stimulus constant by keeping the temperature and the flow rate of cold air constant. The cold sensation presentation device 1 may keep the intensity of the cold stimulus constant by keeping the temperature of the cold air constant, or may keep the intensity of the cold stimulus constant by keeping the flow rate of the cold air constant.

The cold sensation presentation device 1 may keep the intensity of the cold stimulus constant by keeping both the temperature of the cold air and the flow rate of the cold air constant.

When the flow rate of the cold air jet and the temperature of the cold air jet temporally change, the subject may have a feeling of discomfort with the tactile sense due to the change in the cold air jet. For example, temporal change in flow rate of cold air jet may strongly stimulate rapidly adapting tactile receptors. In this case, a tactile sense that is not originally desired to be given to the subject may occur in the subject.

On the other hand, the cold sensation presentation device 1 of the present embodiment combines warm and cold stimuli by convection and radiant heat, and provides a warm or cold sensation while keeping a cold air jet constant. As a result, according to the cold sensation presentation device 1, the tactile sense caused by cold air can be intentionally controlled independently of the warm or cold sensation.

That is, the cold sensation presentation device 1 of the present embodiment achieves continuous heat sensation in a contactless manner without substantial temperature change and a feeling of discomfort of tactile sensation.

More specifically, the cold sensation presentation device 1 of the present embodiment keeps each of the intensity of the cold stimulus constant by keeping the temperature (e.g., room temperature) of the environmental air before compressed, the pressure of the compressed air, the flow rate of the compressed air, the cold air rate of the vortex tube 16, and the flow rate of the cold air 14 discharged from the presentation unit 13 (hereinafter, also described as a flow rate of the presentation unit 13) constant. Here, the cold air rate of the vortex tube 16 refers to a proportion of the flow rate of cold air discharged from the vortex tube 16 to the flow rate of the compressed air 15 supplied to the vortex tube 16.

In the experiments of the present embodiment, the environmental air before compression (room temperature) was “24° C.”, the pressure of the compressed air was “0.6 Pa”, the flow rate of the compressed air was “0.0012 m³/s”, the cold air rate of the vortex tube 16 was “75%”, the flow rate of the presentation unit 13 was “0.000375 m³/s” (when temporal change rate of temperature −0.08 m/s), “0.000447 m³/s” (when temporal change rate of temperature was −0.12 m/s), “0.000532 m³/s” (when temporal change rate of temperature was −0.16 m/s), “0.000625 m³/s” (when temporal change rate of temperature was −0.20 m/s), and “0.000673 m³/s” (when temporal change rate of temperature was −0.24 m/s), and were each kept constant.

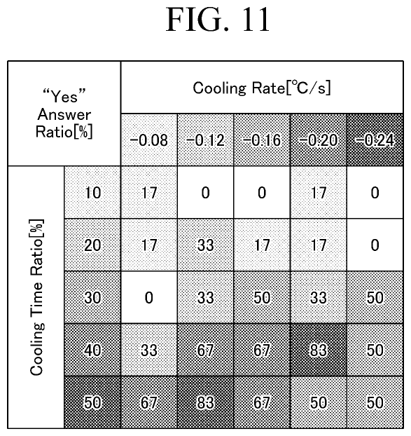

Specific experimental results will be described with reference to FIGS. 10 and 11.

FIG. 10 is a diagram showing an example of an experimental result by the cold sensation presentation device 1 of the present embodiment.

FIG. 11 is a diagram showing an example of an experimental result in a case of temporally changing a cold air jet.

In FIGS. 10 and 11, the horizontal axes represent the temporal change rate in temperature (° C./s) in the skin, and the vertical axes represent the proportion (%) of the cold stimulus time in the warm stimulus time and the cold stimulus time. The numerical values in the tables indicate the proportion of the subjects who answered “cold sensation is sustained”. That is, the numerical values in the tables indicate the proportion of the subjects having only a cold sensation and not having a feeling of discomfort in tactile sense or having a warm sensation. The larger the numerical values in the tables, the more the cold sensation is felt sustained, and the less discomfort there is.

In the case shown in FIG. 11, that is, in the case where the cold air jet temporally changes, the proportion of answering “cold sensation is sustained” was 83% at the maximum and 0% at the minimum.

On the other hand, in the case shown in FIG. 10, that is, in the case where there was no temporal change in the cold air jet and the intensity of the cold stimulus was kept constant, the proportion of answering “cold sensation is sustained” was 100% at the maximum and 7% at the minimum. In particular, in the combination of the condition of the temporal change rate (horizontal axes) in temperature in the skin and the condition of the proportion (vertical axes) of cold stimulus time, the combination in which the proportion of answering “cold sensation is sustained” exceeds 70% was 2 cells in the case of FIG. 11, whereas it was 15 cells in the case of FIG. 10.

The experimental results shown in FIGS. 10 and 11 indicate that the proportion of answering “cold sensation is sustained” was higher when the cold air jet did not temporally change than when the cold air jet temporally changed.

These experimental results indicate that the cold sensation presentation device 1 of the present embodiment can contactlessly give continuous heat sensation to the subject while reducing the feeling of discomfort of the warm sensation and the tactile sense.

### Example of Changing Cold Stimulus

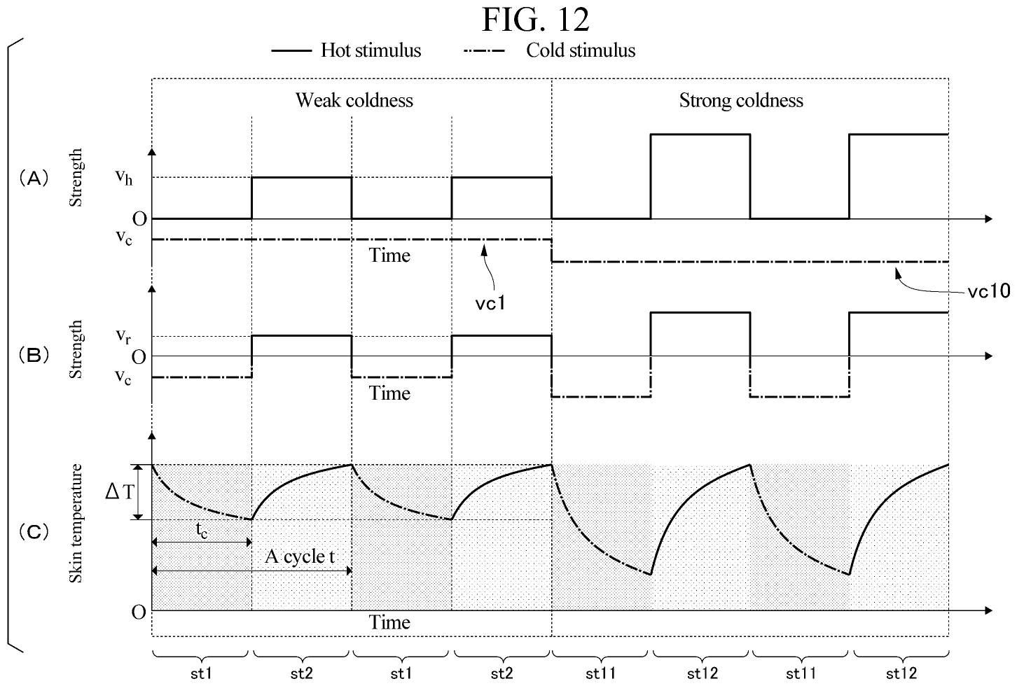

FIG. 12 is a diagram showing a variation of control by the control unit 30 of the present embodiment. In the example shown in FIG. 8 described above, the case where the temporal change rate of the target temperature is not changed (i.e., made constant) on the skin of the subject has been described. FIG. 12 is different from the case shown in FIG. 8 in that the temporal change rate of the target temperature is changed.

FIG. 12[A] individually shows a presentation pattern of the cold stimulus by the cold stimulus section 10 and a presentation pattern of the warm stimulus by the warm stimulus section 20.

FIG. 12[B] shows a result of combining the presentation pattern of the cold stimulus by the cold stimulus section 10 and the presentation pattern of the warm stimulus by the warm stimulus section 20.

As a result of presentation of the cold stimulus and the warm stimulus shown in FIG. 12[A] and FIG. 12[B], the temperature of the skin changes as shown in FIG. 12[C].

FIGS. 8[A] and 8[B] show the cold stimulus presentation pattern and the warm stimulus presentation pattern as individual graphs. On the other hand, FIGS. 12[A] and 12[B] show the cold stimulus presentation pattern and the warm stimulus presentation pattern as one graph. In FIGS. 12[A] and 12[B], origins O of the vertical axes of the graphs are defined as the stimulus intensity 0 (zero), and with the origin O as a boundary, the positive direction of the vertical axes indicates a direction in which the intensity of the warm stimulus is stronger, and the negative direction of the vertical axes indicates a direction in which the intensity of the cold stimulus is stronger.

In this example, the control unit 30 performs control with a weak cold state including the cold stimulus state st1 and the warm stimulus state st2 and a strong cold state including a cold stimulus state st11 and a warm stimulus state st12. The control unit 30 makes the intensity (vc10) of the cold stimulus in the strong cold state stronger than the intensity (vc1) of the cold stimulus in the weak cold state.

The control unit 30 changes the temporal change rate of the target temperature from the weak cold state to the strong cold state. The control of the control unit 30 in the weak cold state (i.e., the cold stimulus state st1 and the warm stimulus state st2) shown in FIG. 12 is similar to that in the case shown in FIG. 8, and thus the description thereof is omitted.

The control unit 30 keeps a discharge temperature and a discharge amount of the cold air 14 by the cold stimulus section 10 constant in the cold stimulus state st11 and warm stimulus state st12 in the strong cold state. The control unit 30 decreases the emission amount of the light 22 by the warm stimulus section 20 (weakens the light 22) in the cold stimulus state st11, and increases the emission amount of the light 22 by the warm stimulus section 20 (strengthens the light 22) in the warm stimulus state st12.

Since the warm stimulus is not presented and the cold stimulus is presented in the cold stimulus state st11, the result of combining the warm stimulus and the cold stimulus is the intensity of only the cold stimulus.

Since the warm stimulus and the cold stimulus are simultaneously presented in the warm stimulus state st12, in the result of combining the warm stimulus and the cold stimulus, a temperature decrease due to the cold stimulus is canceled and a small temperature increase remains.

Thus, even in a case of changing the temporal change rate of the target temperature, the control unit 30 presents a cold sensation by repeating the cold stimulus state and the warm stimulus state in a state of keeping the intensity of the cold stimulus constant. According to the cold sensation presentation device 1 of the present embodiment thus configured, it is possible to contactlessly give continuous heat sensation to the subject while reducing a feeling of discomfort caused by warmness and tactile sensation.

Note that a part of the cold sensation presentation device 1 in each embodiment described above, for example, the control unit 30 and the like may be implemented by a computer. In that case, a program for implementing this function may be recorded in a computer-readable recording medium, and the program recorded in this recording medium may be read and executed by a computer system to implement the function. Note that the “computer system” mentioned here is a computer system incorporated in the cold sensation presentation device 1, and includes hardware such as an operating system (OS) and peripheral devices.

In addition, the “computer-readable recording medium” refers to a portable medium such as a flexible disk, a magneto-optical disk, a ROM, or a CD-ROM, and a storage device such as a hard disk incorporated in a computer system. In addition, the “computer-readable recording medium” may include a recording medium that dynamically stores a program for a short period of time, such as a communication wire when the program is transmitted via a network such as the Internet or a communication line such as a telephone line, and a recording medium that stores a program for a fixed period of time, such as volatile memory inside a computer system that serves as a server or a client in the above-mentioned case. Further, the above-described program may be a program for achieving some of the above-described functions, or may be a program that can achieve the above-described functions in combination with a program that is already recorded in the computer system.

Although one embodiment of the present invention has been described above in detail with reference to the drawings, specific configurations are not limited to those described above, and various changes in design or the like may be made within the scope that does not depart from the gist of the invention.

### REFERENCE SIGNS LIST

1 Cold sensation presentation device,
10 Cold stimulus section,
11 Cold air generation unit,
12 Flow rate control unit,
13 Presentation unit,
20 Warm stimulus section,
21 Light source,
30 Control unit

1. A cold sensation presentation device comprising:
a cold stimulus section configured to contactlessly give a cold stimulus to a skin in a non-contact manner using cold air;
a warm stimulus section configured to contactlessly give a warm stimulus to a skin in a non-contact manner; and
a control unit configured to control the cold stimulus by the cold stimulus section and the warm stimulus by the warm stimulus section, wherein the control unit performs control so as to present a cold sensation by alternately repeating a cold stimulus state where an intensity of the warm stimulus is relatively small and a warm stimulus state where an intensity of the warm stimulus is relatively large in a state of keeping an intensity of the cold stimulus constant while continuously presenting the cold stimulus from the cold stimulus section.

2. The cold sensation presentation device according to claim 1, wherein by alternately repeating the cold stimulus state where an intensity of the warm stimulus is relatively small and the warm stimulus state where an intensity of the warm stimulus is relatively large in a state of keeping an intensity of the cold stimulus constant while continuously presenting the cold stimulus from the cold stimulus section, the control unit causes a temporal change of a skin temperature in the cold stimulus state and a temporal change of a skin temperature in the warm stimulus state to be different from each other.

3. The cold sensation presentation device according to claim 1, wherein after a predetermined time from a state of keeping an intensity of the cold stimulus constant while continuously presenting the cold stimulus, the control unit changes an intensity of at least one of the warm stimulus and the cold stimulus.

4. The cold sensation presentation device according to claim 1, wherein the control unit keeps an intensity of the cold stimulus constant by keeping a temperature and a flow rate of cold air constant.

5. The cold sensation presentation device according to claim 1, wherein the control unit temporally changes an intensity of the warm stimulus from a relatively small state to a relatively large state in the warm stimulus state.

6. The cold sensation presentation device according to claim 1, wherein the control unit temporally changes an intensity of the cold stimulus from a relatively small state to a relatively large state in the warm stimulus state.

7. The cold sensation presentation device according to claim 1, wherein the control unit ends cold sensation presentation by gradually decreasing an intensity of the cold stimulus and an intensity of the warm stimulus or gradually decreasing presentation time of the cold stimulus and presentation time of the warm stimulus.

8. The cold sensation presentation device according to claim 1, wherein among conduction, convection, and radiation, which are types of heat transfer methods, a type with which the cold stimulus section gives the cold stimulus and a type with which the warm stimulus section gives the warm stimulus are different from each other.

9. A non-transitory computer readable medium holding instructions for causing a computer included in a control unit configured to control a cold stimulus state by a cold stimulus section and a warm stimulus state by a warm stimulus section of a cold sensation presentation device including the cold stimulus section that gives a cold stimulus to a skin in a non-contact manner using cold air and the warm stimulus section that gives a warm stimulus to a skin in a non-contact manner to execute presenting a cold sensation by alternately repeating a cold stimulus state where an intensity of the warm stimulus is relatively small and a warm stimulus state where an intensity of the warm stimulus is relatively large in a state of keeping an intensity of the cold stimulus constant while continuously presenting the cold stimulus from the cold stimulus section.
